(19) United States
(12) Patent Application Publication
(10) Pub. No.: US 2005/0165557 A1
(43) Pub. Date: Jul. 28, 2005

(54) Framework for a biopharmaceutical value chain

(76) Inventors: Lance Anthony Ladic, Monmouth Junction, NJ (US); David Rapaport, Marlboro, NJ (US); Rainer Michelis, East Windsor, NJ (US)

Correspondence Address: Siemens Corporation;Intellectual Property Department, 170 Wood Avenue South, Iselin, NJ, 08830, US

(21) Appl. No.: 11/043,392

(22) Filed: Jan. 25, 2005

Related U.S. Application Data
(60) Provisional application No. 60/539,638, filed on Jan. 28, 2004.

(51) Int. Cl. G06F 17/60; G06F 19/00; G01N 33/48; G01N 33/50

(52) U.S. Cl. 702/19; 705/3

(57) ABSTRACT

An information management framework includes a network of connected processors, an application data server connected to the network, and an infrastructure for interfacing with the application data server. The framework includes a first client accessing the application data server through the infrastructure and making drug candidate data available on the application data server via the network, and a second client for conducting drug candidate research via the network against the drug candidate data.

This application claims priority to U.S. Provisional Application Ser. No. 60/539,638, filed on Jan. 28, 2004, which is herein incorporated by reference in its entirety.

### BACKGROUND OF THE INVENTION

1. Technical Field

The present invention relates to pharmaceutical development, and more particularly to a system and method for a discovery-to-Phase-2 framework for a biopharmaceutical value chain.

2. Discussion of Related Art

A problem facing the biopharmaceutical industry is the careful selection of drug candidates. It is estimated that the cost to develop a single, novel compound into a marketable drug is approximately $800M (Tufts Center for the Study of Drug Development) with up to a 15-year development timeline from discovery to U.S. Food and Drug Administration (FDA) approval (Ernst & Young, 2000). Furthermore, the FDA's drug approval process has been slowing. The number of new drugs approved by the FDA fell to 17 in 2002, compared with a 1996 peak of 53 (FDA, 2003).

Consolidation of the industry also plays a role in the selection process of new drug candidates. Large biopharmaceutical enterprises need a large return on investment (ROI) to cover high internal development expenses. This is one reason that the industry is growing more and more risk-averse. “De-risking” has become a term used in the pharmaceutical industry when referring to the concept of investing only in those drugs that have the best chance for FDA approval.

Smaller biotech companies and academic centers, which are an integral part of the drug discovery pipeline, function in the early stages of biopharmaceutical value chain (see FIG. 1). Academic research centers often lack the internal resources to efficiently develop and promote the results of novel, early preclinical research. Furthermore, the biotech companies, particularly start-ups, may lack the internal resources and technological infrastructure to move drug candidates into a position where the candidate would be attractive to larger biopharmaceutical companies for investment.

The declining research and development (R&D) productivity of the larger pharmaceutical companies, coupled with the failure of mergers to generate better drug development pipelines, has caused many pharmaceutical companies to look for collaborations with biotech and academia to fill these gaps. At the same time, a lack of internal start-up capital and low venture capital investment has caused drug candidates developed by smaller biotech companies and academic research centers to remain bottlenecked in the early stages of the biopharmaceutical value chain, prior to Phase 2, the point near which the interest of larges pharmaceutical companies is piqued.

Referring to FIG. 1, Phase 1 trials are clinical trials conducted to evaluate the safety of a drug or therapy; how a drug should be administered (e.g., oral, injection) and evaluation of dose levels. Phase 2 trials are conducted to further evaluate the safety of a drug or therapy and to evaluate drug efficacy. In addition, optimal dose levels are determined. Phase 3 trials are conducted to confirm the efficacy of a new drug or therapy.

Two recent examples that illustrate the willingness of large pharmaceutical companies to invest in drugs that have made it to early phase clinical trials are the relationships between Aventis and Regeneron, and Amgen and Biovitrum (NY Times, Sep. 9, 2003). Aventis, a large French pharmaceutical company, agreed to pay up $125 million up front, and possibly up to $510 million, to license a cancer drug candidate that is in Phase 1 of clinical trials from tiny Regeneron Pharmaceuticals located in Tarrytown, N.Y. Amgen, the world's largest biotechnology company, has agreed to pay Biovitrum, a small Swedish company, $86.5 million up front, followed by up to $400 million, for the rights to a novel diabetes drug candidate, which is in early Phase 2 of clinical trials.

Therefore, a need exists for a system and method for streamlining and efficiently/effectively moving drug candidates from the early phases of basic research and discovery to Phase 2 of the clinical trial cycle.

### SUMMARY OF THE INVENTION

According to an embodiment of the present disclosure, an information management framework comprises a network of connected processors, an application data server connected to the network, an infrastructure for interfacing with the application data server, a first client accessing the application data server through the infrastructure and making drug candidate data available on the application data server via the network, and a second client for conducting drug candidate research via the network against the drug candidate data.

The information management framework comprises a hardware module providing hardware to the first client and/or the second client.

The information management framework comprises a software module providing applications to the first client and/or the second client. The software module comprises at least one or a data mining application, a simulation application, a modeling application, an imaging application, a decision support system, and a network security application.

The information management framework comprises an advisory board module, wherein the advisory board module screens drug candidate data provided by the providing client for inclusion in the information management framework.

The infrastructure comprises a laboratory information management system. The infrastructure comprises an e-commerce module for facilitating a transaction between the first client and the second client.

According to an embodiment of the present disclosure, a method for promoting a drug candidate comprises providing drug candidate data to a library information management system, creating an abstract of the drug candidate data, providing accessing to the abstract through an application service provider, the application service provider coupled to a server, and facilitating a financial transaction between a provider of the drug candidate data and an investor.

The method includes assigning a value to the drug candidate data.

The financial transaction includes a sale of the drug candidate data to the investor. The financial transaction includes a promise to pay a royalty to the provider of the drug candidate data by the investor in exchange for the drug candidate data.

The method includes aggregating the drug candidate data with data in the library information management system.

The method comprises modifying the drug candidate data in compliance with privacy regulations.

The method includes encrypting the drug candidate data. The financial transaction includes providing the investor with a key for decrypting the drug candidate data.

According to an embodiment of the present disclosure, an infrastructure for promoting a drug candidate comprises a communications network coupled to a plurality of clients, a server providing an abstract of drug candidate data via the communications network, a library information management system for processing the abstract of the drug candidate data, and a commerce module for purchasing drug candidate data from the provider.

The library information management system enables at least one of grouping abstracts, tracking abstract updates, and viewing abstracts. The library information management system enables at least one of grouping purchased drug candidate data, tracking purchased drug candidate data updates, and viewing purchased drug candidate data.

### BRIEF DESCRIPTION OF THE DRAWINGS

Preferred embodiments of the present invention will be described below in more detail, with reference to the accompanying drawings:

FIG. 1 is an illustration of a biopharmaceutical chain;

FIG. 2 is an illustration of a system according to an embodiment of the present disclosure;

FIG. 3 is an illustration of discovery-to-phase-2 value drivers according to an embodiment of the present disclosure;

FIG. 4 is an illustration of a discovery-to-phase-2 framework in a biopharmaceutical value chain according to an embodiment of the present disclosure;

FIG. 5 is an illustration of a network according to an embodiment of the present disclosure;

FIG. 6A is an illustration of a product cycle according to an embodiment of the present disclosure;

FIG. 6B is a flow chart of a method for promoting a product through clinical trials according to an embodiment of the present disclosure; and

FIG. 7 is a flow chart of a method for promoting a product from discovery according to an embodiment of the present disclosure.

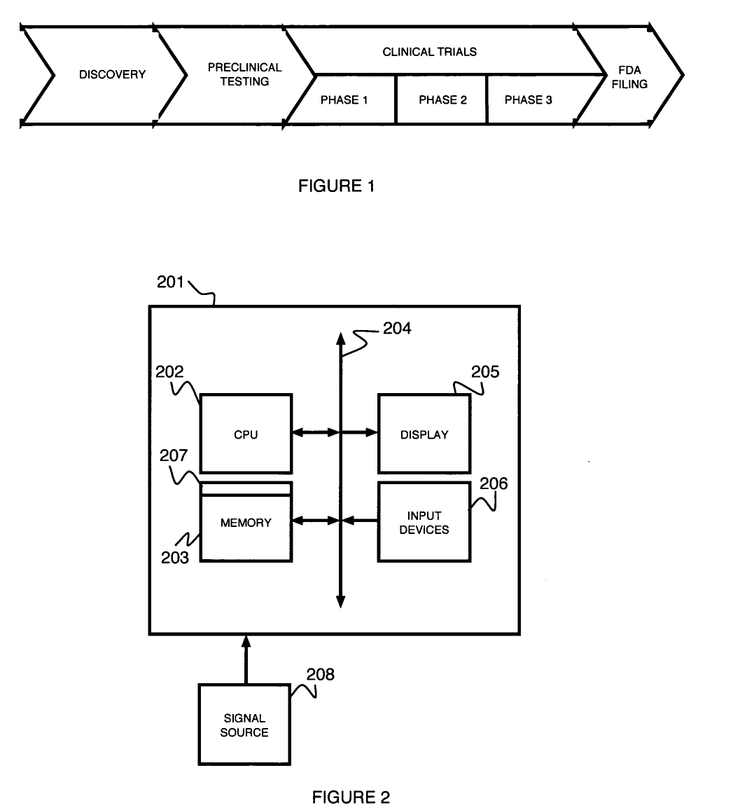

### DETAILED DESCRIPTION OF PREFERRED EMBODIMENTS

According to an embodiment of the present disclosure, a system and method facilitates the promotion of drug candidates from discovery to Phase 2. A shortened timeframe reduces development costs and increases the return on investment (ROI) of the entire biopharmaceutical chain.

It is to be understood that elements of the present invention, e.g., clients, servers, methods, etc., may be implemented in various forms of hardware, software, firmware, special purpose processors, or a combination thereof. In one embodiment, the present invention may be implemented in software as an application program tangibly embodied on a program storage device. The application program may be uploaded to, and executed by, a machine comprising any suitable architecture.

Referring to FIG. 2, according to an embodiment of the present disclosure, a computer system 201, such as a client or server, for implementing the present invention can comprise, inter alia, a central processing unit (CPU) 202, a memory 203 and an input/output (I/O) interface 204. The computer system 201 is generally coupled through the I/O interface 204 to a display 205 and various input devices 206 such as a mouse and keyboard. The support circuits can include circuits such as cache, power supplies, clock circuits, and a communications bus. The memory 203 can include random access memory (RAM), read only memory (ROM), disk drive, tape drive, etc., or a combination thereof. The present invention can be implemented as a routine 207 that is stored in memory 203 and executed by the CPU 202 to process the signal from the signal source 208. As such, the computer system 201 is a general purpose computer system that becomes a specific purpose computer system when executing the routine 207 of the present invention.

The computer platform 201 also includes an operating system and micro instruction code. The various processes and functions described herein may either be part of the micro instruction code or part of the application program (or a combination thereof) which is executed via the operating system. In addition, various other peripheral devices may be connected to the computer platform such as an additional data storage device and a printing device.

It is to be further understood that, because some of the constituent system components and method steps depicted in the accompanying figures may be implemented in software, the actual connections between the system components (or the process steps) may differ depending upon the manner in which the present invention is programmed. Given the teachings of the present invention provided herein, one of ordinary skill in the related art will be able to contemplate these and similar implementations or configurations of the present invention.

The completion of the Human Genome Project in 2003 has heralded the start of a new era in medicine that is reaping the rewards of biotechnology. More than 325 million people worldwide have been helped by the more than 155 biotechnology drugs and vaccines approved by the FDA such as Herceptin for metastatic breast cancer, Procrit for chemotherapeutic-induced anemia, and Rituxan for B-cell non-Hodgkin's lymphoma. Furthermore, there are more than 370 biotech drug products and vaccines currently in clinical trials targeting more than 200 genetically-linked diseases, including various cancers, Alzheimer's disease, heart disease, diabetes, multiple sclerosis, AIDS (Acquired Immune Deficiency Syndrome) and arthritis.

These advances related to biotechnology will have an impact on healthcare businesses including medical equipment and healthcare information technology (IT) areas. In the medical equipment market, a need exists for an integration of clinical data emerging from research with hardware such as imaging modalities, e.g., new contrast agents for molecular imaging. There is also a need for the development of equipment for acquiring and communicating clinical data, e.g., optical imaging, and point-of-care testing devices. In the area of healthcare IT, opportunities for product differentiation will arise through the acquisition, integration and post-processing of data from various biotech sources, such as genomics, proteomics, metabolomics, with existing medical data.

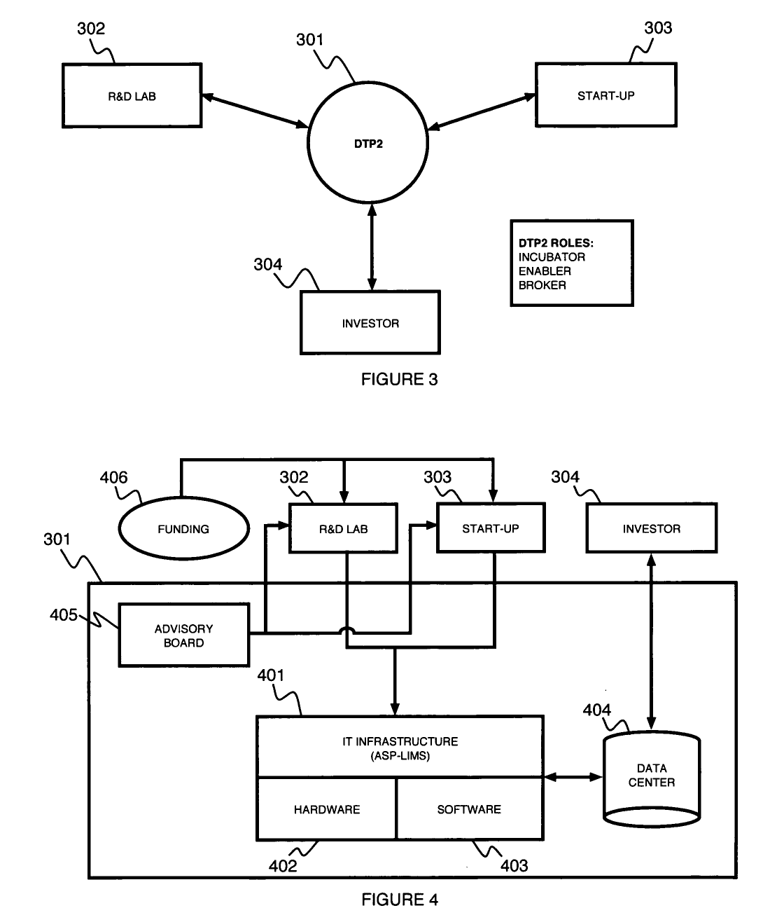

According to an embodiment of the present disclosure, a Discovery-to-Phase-2 (DTP2) framework 301 is positioned as an incubator, enabler and broker between R&D labs 302, start-up companies 303 and investors 304, such as pharmaceutical enterprises (see FIG. 3). The framework 301 integrates drug candidate clinical data, supplied by R&D labs 302 and start-up companies 303, and investors 304.

The R&D lab 302 adds to the framework pre-clinical data, which is aggregated in a data center of the framework 301. The R&D lab 302 provides a renewable source of technology and methods. Further, the R&D lab 302 adds credibility and domain expertise to the framework 301.

Start-up firm 303 provides supporting data for drug candidates and pre-clinical data to be stored in the data center. The credibility and domain expertise of the start-up is also a value added to the framework.

The investor 304 adds royalty fees for promising drug candidates that have passed Phase 2 trials. The investor may also provide supporting data. The investor 304 may finance the framework 301 as an incubator for drug candidates. Where the investor 304 is a pharmaceutical enterprise or other entity in the pharmaceutical field, the involvement of the investor 304 adds credibility and industry exposure for the framework 301.

Referring to FIG. 4, the framework 301 comprises an IT infrastructure backbone module 401 including complementary hardware 402 and software modules 403. The framework 301 provides access to relevant clinical data from an Application Service Provider (ASP)/data center module 404. The framework 301 has the capability to provide data archival and management services through the ASP/data center module 404. Furthermore, the framework 301 facilitates e-commerce transactions to provide investors 304 with Phase 2 and other pre-clinical data through the ASP/data center module 404. The framework 301 can effectively streamline and speed up the flow of drug candidates through the biopharmaceutical value chain and enable investors 304, such as pharmaceutical enterprises, to make an informed, lower risk decision as to where to place investment dollars. The framework 301 further comprises sources of funding 406 for R&D 302 and start-ups 303. Funding sources 406 may include investors 304, government agencies/programs, etc.

The infrastructure module 401 provides electronic data brokering services. The infrastructure module 401 includes a data acquisition hardware module 402, data management and analysis software 403, and the ASP/data center module 404 for archiving and storage.

The IT infrastructure module 401 supports an ASP-LIMS (Application Service Provider-delivered Laboratory Information Management System) for the biopharmaceutical value chain, similar to systems such as Simatic IT Unilab or Soarian™. The ASP-LIMS provides, for example, hardware infrastructure for the ASP/data center 404, libraries of related workflow modules, applications specific to processing laboratory data, embedded DSS (Decision Support System) software, and a data warehouse, e.g., ASP/data center 404 for storing customer data in encrypted format for backup purposes and/or to facilitate e-commerce. ASP hardware includes one or more servers, and network connectivity devices. The DSS includes a database, hardware such as a server, and a DSS application for conducting business analyses.

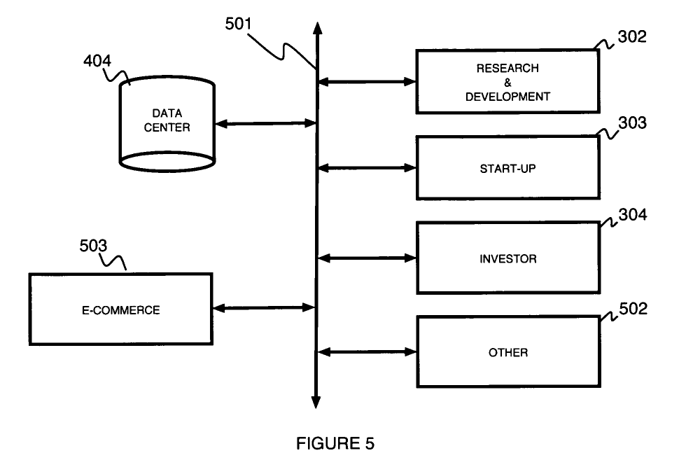

FIG. 5 illustrates a network 501 supporting LIMS interactions between a plurality of clients 302-304 and 502 and a LIMS ASP/data center 404. The LIMS ASP/data center module 404 cooperates with an e-commerce module 503 of the infrastructure 401 to provide drug candidate data to purchasing clients. The infrastructure 401 constructs libraries of workflow modules where each module corresponds to a particular laboratory workflow, e.g., for drug discovery workflows may include drug interactions, dosage, and efficacy. The infrastructure 401 and LIMS ASP/data center 404 may be extended to other forms of laboratory/clinical data.

The infrastructure 401 has the capacity to interface with specific hardware 402 for data acquisition using open communication standards; integrate integral software components 403 for data management and analysis, also using open standards; connect with the ASP/data center 404; and adhere to regulatory standards, e.g., FDA CFR 22 Part 11. Proprietary systems may also be used.

The infrastructure 401 supports e-commerce hosting and/or brokering of drug candidate data. Clients may generate abstracts of drug candidate data, e.g., via a software application supplied by the ASP/data center 404, to be hosted. For example, the software application permits intelligent tagging of the data that provides detailed information about the data and an associated financial value. Additionally, the software application on the client side allows transmission of a data abstract from the client's data repository, e.g., an R&D client's research, to the e-commerce server hosted by the ASP/data server 404 as well as facilitate a secure transmission of data between the client and an external party or another client such as a pharmaceutical enterprise in the event of a financial transaction. The software application also allows consolidation of the data abstracts from multiple clients into a master abstract and the ability to associate an individual data item with an individual client.

To illustrate, the ASP/data center 404 may host a web site that is accessible by a plurality of clients. Thus, an investor client can search data abstracts hosted by the ASP/data center 404. The data abstract provides the investor client with an abstract view of the drug candidate data, which may be stored on the providing client side. The data abstract may be from a single client or represent a master data abstract that has a consolidated version of data abstracts from a plurality of clients. If investor client locates a piece of data that it wishes to purchase, the LIMS facilitates a secure transmission between the investor client and the appropriate client providing the data. In this way, the LIMS serves as the information broker between clients.

In the event that the data refers to a physical object being offered for sale, e.g., a custom-designed biochip or a drug sample, and a transaction request is made, the LIMS facilitates the order request on behalf of the pharmaceutical enterprise client to the client providing the object, e.g., provide the billing/shipping information to the client providing the object. Thus, the infrastructure 401 is not limited to e-commence of electronic products that can be electronically transferred to the customers in a secured transmission medium; it also encompasses the sending of physical products to clients.

Data from the clients is stored in an encrypted format in the ASP/data center 404 and made available through the LIMS. The LIMS includes tools for grouping abstracts, tracking abstract updates, and viewing abstracts, etc. The tools may also be used with purchased drug candidate data. A pharmaceutical enterprise as buyer used the tools provided by the LIMS for searching the data abstract(s) stored in the ASP/data center 404. If an item of interest is found and a financial transaction is requested, the desired data is retrieved in an encrypted format from the ASP/data center 404 and sent to the pharmaceutical enterprise as buyer. A notification would be sent to the selling client, e.g., a start-up, requesting that a decryption key(s) specific to the data being purchased be sent to the buyer. The buyer unlocks the data on their side into an unencrypted readable/usable form. Since the selling client only sends key(s) to unlock the data, which could be embedded into an email, reducing the amount of bandwidth used on the selling client's side.

The data abstract provided by each client includes data output of an overall workflow and data generated at intermediate workflow steps, as these may also possess value. Examples of data output of that may possess financial value may include, for example, generalized demographics data statistics, associated with other data objects, drug efficacy, drug chemistries, and imaging data.

The data may be stripped of all personal identifiers and remain generic, in accordance with statutes, regulations, etc., for example, HIPAA (Health Insurance Portability and Accountability Act).

The hardware module 402 for data acquisition may utilize open standards to communicate with third-party hardware. Hardware components 402 include, but are not limited to, diagnostic and therapeutic imaging hardware, biochips, point-of-care devices.

Diagnostic and therapeutic imaging centers, which may operate as independent, for-profit institutions, may be shared among the companies (academia/biotech) being incubated in the framework 301 as well as the pharmaceutical enterprises. Hardware housed in these facilities includes molecular/optical imaging using plug-in technology, small animal imaging, and live Cell imaging (e.g. 4D microscopy).

Biochips and associated reader/analysis devices include DNA Chips, protein chips and lab-on-a-chip technologies. Point-of-Care (POC) devices may be provided as hardware 402 and may include, for example, readers/analyzers and remote monitors.

The software module 403 (e.g., data management, analysis, e-commerce) is a package of integrated software applications needed to manage and objectively analyze data and supply information on all R&D data up to Phase 2 of the clinical trial process. This includes, but is not limited to, workflows, security (e.g., encryption, HIPAA compliance), and bioinformatics.

Bioinformatics includes biomarker identification, markers that uniquely identify disease at the molecular level, through data analysis/mining methods. Data analysis/mining methods may be used for post-processing of data acquired from hardware such as mass spectrometry, nuclear magnetic resonance (NMR) and biochips. The software components for performed image visualization and analysis for decision support systems/computer aided diagnosis, and simulation and modeling.

The ASP/data center module 404 supports the remote hosting of applications. These applications may be accessed by clients via network connections 501 (see FIG. 5). Clients include, for example, the R&D labs 302, pharmaceutical enterprises 304, and others 502 such as government funding agencies having appropriate permissions for accessing the framework. Multiple network connectivity options are provided, including high-speed Internet, intranet, and Virtual Private Networks (VPNs). Services provided by the ASP/data center 404 include, for example, database administration, production control, network management, data security, non-disruptive maintenance, availability and performance management, capacity planning, automated operations, systems programming, storage, backup, and disaster recovery, change management, and data mining tools.

The ASP/data center module 404 archives and store R&D data. The ASP/data center module 404 allows access to pertinent clinical and financial healthcare data. The clinical and financial healthcare date may be utilized in patient selection/recruitment for clinical trials. The system utilizes this information with the biomarker identifier to more accurately find appropriate patient candidates for clinical trial testing.

The framework 301 implements an advisory board module 405. The advisory board module 405 includes internal and external domain experts. The external experts should be leaders in the biopharmaceutical market, preferably with ties to pharmaceutical companies. Their function includes participation in a screening of drug candidates for incubation in the DTP2 framework 301, and an evaluation of drug candidates and associated data to be brokered via the framework 301.

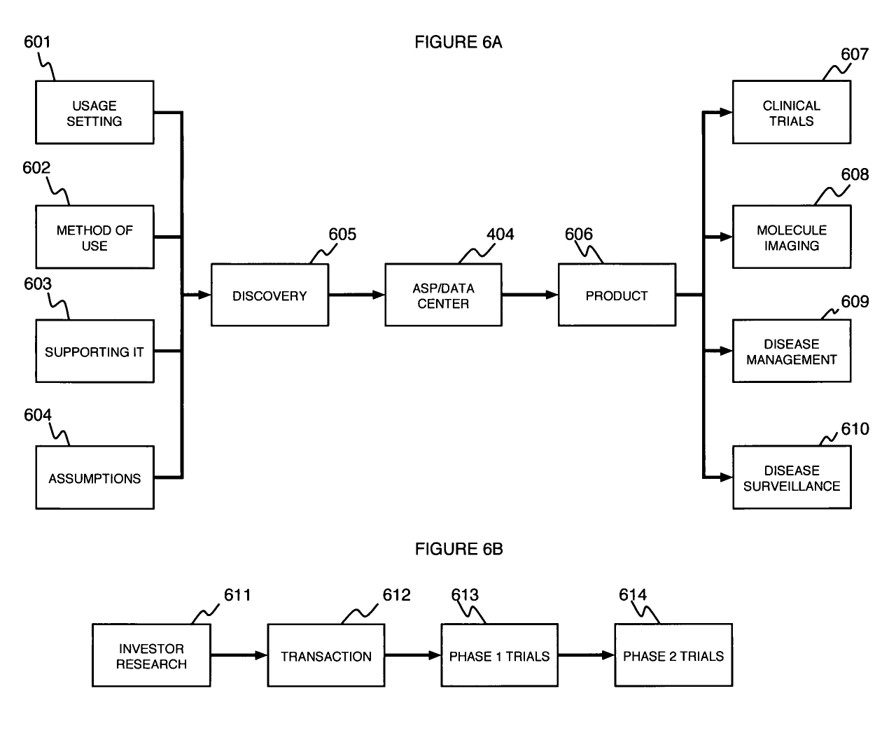

FIG. 6A illustrates the application of a product such as a drug candidate or biochip within the framework 301. Data including usage settings 601, method of use 602, supporting IT 603 and assumptions 604 are used towards discovery 605, e.g., biomarker identification or drug candidate discovery, within the province of the R&D lab or start-up. The data is stored on or made available to the ASP/data center 404. The ASP/data center 404 incorporates the data with other collected data, including disease states, drug sensitivity, drug efficacy, and toxicity. The ASP/data center provides hardware and software for development of a product 606, e.g., lab-on-chip. The product 606 may be applied to clinical trials 607, imaging 608, disease management 609, disease surveillance 610, etc. Within the clinical trials 607, investors may review the data 601-604 supporting the biomarker 605 and select to purchase data and/or the lab-on-chip 606 or finance clinical trials.

Referring to FIG. 6B, an investor with access to the ASP/data center 404 researches 611 data provided by a providing client. The investor may select to purchase data, products, know-how, etc., from the providing client. The transaction 612 may be, for example, a purchase, royalty paying transaction, or finance agreement. With funding from the transaction, the providing client may promote the drug through clinical trials, including Phase 1 and Phase 2. According to terms of the transaction, the investor may promote the drug through the trials.

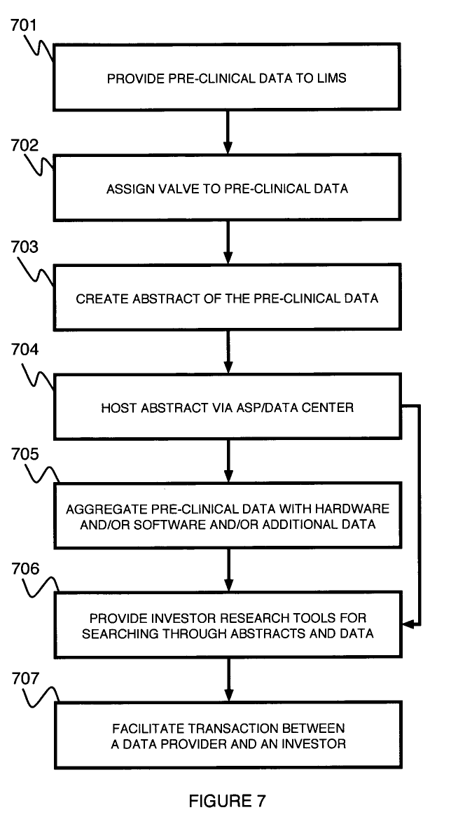

Referring to FIG. 7, an electronic data brokering service provides a mechanism for electronically selling comprehensive drug data sets from all stages of the biopharmaceutical value chain from discovery through Phase 2 of clinical trials. The data sets include all supporting data that complies with FDA regulatory standards. The data is provided to the LIMS system 701. A value is assigned to the data 702, for example, an amount certain or a percentage of revenue from any drug product that attains regulatory approval. The value may be assigned by an advisory board 405 or by the providing client. An abstract of the data is created 703 and made available 704 through the infrastructure hardware and/or software. The provided data may be aggregated with data/services provided by the infrastructure 705, such as simulation and modeling. Research tools, such as data mining applications, are provided to investors for performing research 706. The research tools aid the investor's decision process, e.g., where to place investments. Transactions are facilitated between the data provider and the investor 707, for example, through e-commerce applications.

Having described embodiments for a system and method for a discovery-to-Phase-2 framework, it is noted that modifications and variations can be made by persons skilled in the art in light of the above teachings. It is therefore to be understood that changes may be made in the particular embodiments of the invention disclosed which are within the scope and spirit of the invention as defined by the appended claims. Having thus described the invention with the details and particularity required by the patent laws, what is claimed and desired protected by Letters Patent is set forth in the appended claims.

1. An information management framework comprising:
a network of connected processors;
an application data server connected to the network;
an infrastructure for interfacing with the application data server;
a first client accessing the application data server through the infrastructure and making drug candidate data available on the application data server via the network; and
a second client for conducting drug candidate research via the network against the drug candidate data.

2. The information management framework of claim 1, further comprising a hardware module providing hardware to the first client and/or the second client.

3. The information management framework of claim 1, further comprising a software module providing applications to the first client and/or the second client.

4. The information management framework of claim 3, wherein the software module comprises at least one of a data mining application, a simulation application, a modeling application, an imaging application, a decision support system, and a network security application.

5. The information management framework of claim 1, further comprising an advisory board module, wherein the advisory board module screens drug candidate data provided by the providing client for inclusion in the information management framework.

6. The information management framework of claim 1, wherein the infrastructure comprises a laboratory information management system.

7. The information management framework of claim 1, wherein the infrastructure comprises an e-commerce module for facilitating a transaction between the first client and the second client.

8. A method for promoting a drug candidate comprising:
providing drug candidate data to a library information management system;
creating an abstract of the drug candidate data;
providing an investor access to the abstract through the library information management system, the a library information management system coupled to a server hosting the abstract; and
facilitating a financial transaction between a provider of the drug candidate data and the investor.

9. The method of claim 8, further comprising assigning a value to the drug candidate data.

10. The method of claim 8, wherein the financial transaction includes a sale of the drug candidate data to the investor.

11. The method of claim 8, wherein the financial transaction includes a promise to pay a royalty to the provider of the drug candidate data by the investor in exchange for the drug candidate data.

12. The method of claim 8, further comprising aggregating the drug candidate data with data in the library information management system.

13. The method of claim 8, further comprising modifying the drug candidate data in compliance with privacy regulations.

14. The method of claim 8, further comprising encrypting the drug candidate data.

15. The method of claim 14, wherein the financial transaction includes providing the investor with a key for decrypting the drug candidate data.

16. An infrastructure for promoting a drug candidate comprising:
a communications network coupled to a plurality of clients;
a server providing an abstract of drug candidate data via the communications network;
a library information management system for processing the abstract of the drug candidate data; and
a commerce module for purchasing drug candidate data from the provider.

17. The infrastructure of claim 16, wherein the library information management system enables at least one of grouping abstracts, tracking abstract updates, and viewing abstracts.

18. The infrastructure of claim 16, wherein the library information management system enables at least one of grouping purchased drug candidate data, tracking purchased drug candidate data updates, and viewing purchased drug candidate data.
